(19) United States
(12) Patent Application Publication
(10) Pub. No.: US 2011/0296717 A1
(43) Pub. Date: Dec. 8, 2011

(54) SHOE HAVING A FUNCTIONAL SOLE FOR DEGENERATIVE OSTEOARTHRITIS OF KNEE JOINT

(75) Inventors: Hae Soo Park, Seoul (KR); Hoon Park, Seoul (KR)

(73) Assignee: INTOOS HCN CORPORATION LTD., Seoul (KR)

(21) Appl. No.: 13/201,487

(22) PCT Filed: Feb. 23, 2010
(86) PCT No.: PCT/KR2010/001106

§ 371 Date: Aug. 15, 2011

(30) Foreign Application Priority Data
Feb. 23, 2009 (KR) 10-2009-0014902
Feb. 23, 2009 (KR) 10-2009-0014903

(51) Int. Cl. A61F 5/14

(52) U.S. Cl. 36/140

(57) ABSTRACT

Disclosed therein is a shoe having a functional sole for degenerative osteoarthritis of a knee joint, which includes a sole having at least one of an insole, a midsole and an outsole, characterized in that, in longitudinal section of the insole, the outsole or the midsole formed integrally with one of the insole and the outsole, both sides of an inner face and an outer face thereof are not parallel with each other, and that an angle of one of both sides of the inner face or the outer face to the ground outside the sole is smaller than an angle of the opposed side to the ground outside the sole.

### TECHNICAL FIELD

The present invention relates to a shoe having a functional sole for degenerative osteoarthritis of a knee joint. More particularly, the present invention relates to a shoe having a functional sole for degenerative osteoarthritis of a knee joint, in which, in a longitudinal section of at least one of an insole, an outsole and a midsole of the sole, an angle of one of both sides of an inner face or an outer face to the ground outside the sole is smaller than an angle of the opposed side to the ground outside the sole.

That is, the present invention relates to a shoe having a functional sole for degenerative osteoarthritis of a knee joint, which can correct bow legs or knock knees since both sides are not parallel with each other.

### Background Art

A knee joint is a part of the human body to keep equilibrium in such a way that a human being can keep on his or her feet while bearing the weight of the body with not four feet but two feet. So, the knee joint moves little by little even at the moment the human being cannot be conscious in order to control the balance in such a way that the weight does not get out of the center of the knee joint.

However, the knee joint is a part that degenerative changes occur the most as the human being gets older because the weight is continuously applied thereto. There are lots of kinds of knee joint diseases occurring due to such degenerative changes, external shock or others as follows.

Meniscus damage that meniscus of the knee is cracked due to strenuous exercises, overweight, traumatic injuries, and so on, a damage to knee ligaments occurring due to a strong shock or excessive rotation of joints, chondromalacia patella frequently occurring to young women, and bursitis that bursa of the knee joint gets an infection are diseases frequently occurring at the knee joint.

Degenerative osteoarthritis is a disease that the human being feels pain in the knee when he or she tries to stand up, that the knee is trembled when he or she stands up, that there is a sound when he or she flexes or extends the knee joint, that he or she has an at least first-sized gap between the two knees in a state where he or she stands erect, or that he or she feels a sharp pain when he or she walks down the stairs. Furthermore, people have to be doubtful about the degenerative osteoarthritis if a pain occurring after walk continues for two or three days.

Joints move under load, and especially, power still stronger than the weight is applied to knee joints of the leg by daily actions, such as, standing, walking, climbing stairs, and so on.

In general, the degenerative osteoarthritis occurs mainly to people in their fifties, but osteoarthritis of the knee frequently occurs to people in their forties which is relatively early time. Treatment of osteoarthritis is carried out for the purpose of pain relief and increase of joint flexibility. However, it is difficult to cure the disease completely because it is difficult to remove the fundamental cause of the disease. The pain and the symptom can be relieved through continuous physical therapy, medicinal therapy, weight-loss, and so on if the symptom is slight, and can be relieved through a surgical operation if the symptom is severe.

For example, Knee joint arthroplasty is an operation to remove the knee joint of severe osteoarthritis and transplant a new joint. Patients suffering from osteoarthritis of the knee joint have to continuously get such a treatment for a long time in order to be free from the pain. However, many people endure the pain without a proper treatment due to inconvenience of treatment and burden of expenses.

The weight centerline of the human body passes over the middle between the two feet when the human body is supported by both feet, and hence, a larger load is applied to the inner part of the knee joint rather than the outer part. Moreover, the ground reaction force line passes over the center of foot and the center of pelvis when the human body is supported by only one foot during walking, and hence, the ground reaction force by body-weight is inclined to the inner part of the body from the center of the knee. Accordingly, a compressive load larger than that occurring when the body is supported by both feet is acted to the inner part of the knee.

When the load on the inner knee joint is accumulatively increased, the human being's legs are gradually transformed into bow legs after the forties if he or she does not take lower extremity strengthening exercises. In addition, a range of motion (ROM) of dorsi flexion of the ankle is also reduced, and hence, pitch and stride are also reduced. In relation with frequency of occurrence of osteoarthritis of the knee joint, about 12% of the whole population, about 60% of the number of people over sixty, and about 80% of the number of people over seventy-five are suffering from osteoarthritis of the knee joint.

Particularly, in case of women, they get osteoarthritis of the knee joint in age lower than men due to childbirth, the life pattern that they frequently use the knee joint, physiological changes such as the menses, and so on. Additionally, since women is higher in frequency of occurrence than men, most of the women have bow legs around sixty-five.

Through the clinical observations that a hot spot of degenerative osteoarthritis is the front of the inner part and the legs are started to be gradually transformed into bow legs after forty, degenerative osteoarthritis of the knee joint could be analyzed as a phenomenon occurring by a long biased-concentration of load on the articular surface of the knee. From the above-mentioned result, people can draw a conclusion that dispersion of the biasedly concentrated load can relieve and prevent the pain of degenerative osteoarthritis of the knee joint.

FIGS. 1 to 3 are brief longitudinally sectional views of a convention shoe sole. As shown in FIGS. 1 to 3, the conventional shoe sole includes an inner part and an outer part, which are inclined at right angles to the ground or at the same angle to the ground. Accordingly, it cannot disperse the concentrated load.

In order to solve the above-mentioned problem, in the conventional orthopedic shoemaking, a method of adding a wedge on an inner sole or an outer bottom surface of the shoe sole has been used. Then, the feet and legs lean inwardly according to the slope of the inner sole and the shoe sole when a user steps on the ground with the feet in shoes, and rotational force for moving the knee joint inwardly is caused. However, the conventional method has a problem in that shearing force is generated because the feet slide inwardly due to the inclination of the shoe sole during walking. Furthermore, since the user applies excessive power to the knee and the ankle to prevent slide of the feet, a severe load is acted to the inner parts of the feet and the knees to thereby take a serious turn.

Moreover, the conventional orthopedic shoemaking has several problems in that it must be made as prescribed by a physician to prevent side effects, otherwise serious side effects may occur.

### DISCLOSURE OF INVENTION

### Technical Problem

Accordingly, the present invention has been made in an effort to solve the above-mentioned problems occurring in the prior arts, and it is an object of the present invention to provide a shoe having a functional sole for degenerative osteoarthritis of a knee joint, in which, in a longitudinal section of an insole, an outsole or a midsole formed integrally with the insole or the outsole, an angle of one of both sides of an inner face or an outer face to the ground outside the sole is smaller than an angle of the opposed side to the ground outside the sole.

Another object of the present invention is to provide a shoe having a functional sole for degenerative osteoarthritis of a knee joint, which can correct bow legs or knock knees since both sides of the inner face or the outer face are not parallel with each other.

### Solution to Problem

To achieve the above objects, the present invention provides a shoe having a functional sole for degenerative osteoarthritis of a knee joint, which includes a sole having at least one of an insole, a midsole and an outsole, characterized in that, in longitudinal section of the insole, the outsole or the midsole formed integrally with one of the insole and the outsole, both sides of an inner face and an outer face thereof are not parallel with each other, and that an angle of one of both sides of the inner face or the outer face to the ground outside the sole is smaller than an angle of the opposed side to the ground outside the sole.

In this instance, in longitudinal section of the insole, the outsole or the midsole formed integrally with one of the insole and the outsole, in order to correct bow legs, an angle of an inner side to the ground outside the sole is smaller than an angle of an outer side to the ground outside the sole.

Moreover, in longitudinal section of the insole, the outsole or the midsole formed integrally with one of the insole and the outsole, in order to correct knock knees, an angle of the inner side to the ground outside the sole is smaller than an angle of the inner side to the ground outside the sole.

Furthermore, the insole, the outsole or the midsole formed integrally with one of the insole and the outsole is changeable in inclination angle and thickness according to a wearer's conditions.

Additionally, the outsole includes a recess formed on one side of an upper face thereof, and the insole or the insole and the midsole, which are formed integrally with each other, is inserted into the recess of the outsole.

In addition, the outsole of the sole or the outsole and the midsole, which are formed integrally with each other, includes a projection formed along the outer circumferential surface of the upper face thereof, so that the sole can be joined with the upper smoothly.

Moreover, in case of the insole, the outsole or the midsole formed integrally with one of the insole and the outsole, the angle of the inner side to the ground outside the sole for correcting the bow legs and the angle of the outer side to the ground outside the sole for correcting the knock knees are within a range of 45° to 120°.

Furthermore, the upper is constructed in such a way as to prevent exposure of the insole and the midsole to the outside.

### ADVANTAGEOUS EFFECTS OF INVENTION

The shoe having the functional sole according to the present invention can naturally correct bow legs or knock knees during walking by uniformly dispersing load biasedly applied to the knees to thereby relieve and prevent pains of degenerative osteoarthritis of the knee joint.

Furthermore, the shoe having the functional sole according to the present invention can establish correct gait and body structure through the correction of the bow legs or the knock knees to thereby correct the wearer's general body frame.

### BRIEF DESCRIPTION OF DRAWINGS

The above and other objects, features and advantages of the present invention will be apparent from the following detailed description of the preferred embodiments of the invention in conjunction with the accompanying drawings for understanding of technical ideas of the present invention, and hence, the present invention will not be restricted to the drawings.

FIGS. 1 to 3 are brief longitudinally sectional views of a convention shoe sole.

FIG. 4 is a longitudinally sectional view showing a shoe including a slanted functional sole having an insole and a midsole to cure a degenerative knee disease according to a first preferred embodiment of the present invention.

FIG. 5 is a longitudinally sectional view showing a shoe including a slanted functional sole having a midsole and outsole to cure a degenerative knee disease according to the first preferred embodiment of the present invention.

FIG. 6 is a brief longitudinally sectional view showing couple moment occurring by a sole for a wearer, who has bow legs, according to a preferred embodiment of the present invention.

FIG. 7 is a brief longitudinally sectional view showing couple moment occurring by a sole for a wearer, who has bow legs, according to another preferred embodiment of the present invention.

FIG. 8 is a brief view showing abduction moment of the conventional shoe.

FIG. 9 is a brief view showing abduction moment of the shoe having the insole and the midsole according to the present invention.

FIG. 10 is a brief view showing abduction moment of the shoe having the outsole and the midsole according to the present invention.

FIG. 11 is a brief longitudinally sectional view showing couple moment occurring by a sole for a wearer, who has knock knees, according to a preferred embodiment of the present invention.

FIG. 12 is a brief longitudinally sectional view showing couple moment occurring by a sole for a wearer, who has knock knees, according to another preferred embodiment of the present invention.

FIG. 13 is a longitudinally sectional view of a shoe including a slanted functional sole having an insole and a midsole to cure a degenerative knee disease according to a modification of the first preferred embodiment of the present invention.

FIG. 14 is an exploded perspective view of a sole according to a second preferred embodiment of the present invention.

FIG. 15 is a longitudinally sectional view of the sole according to the second preferred embodiment of the present invention.

FIG. 16 is a longitudinally sectional view of a shoe including a slanted sole to cure a degenerative knee disease according to a third preferred embodiment of the present invention.

FIG. 17 is a longitudinally sectional view of a shoe including a slanted sole to cure a degenerative knee disease according to a modification of the third preferred embodiment of the present invention.

FIG. 18 is a longitudinally sectional view of a shoe including a slanted sole to cure a degenerative knee disease according to a fourth preferred embodiment of the present invention.

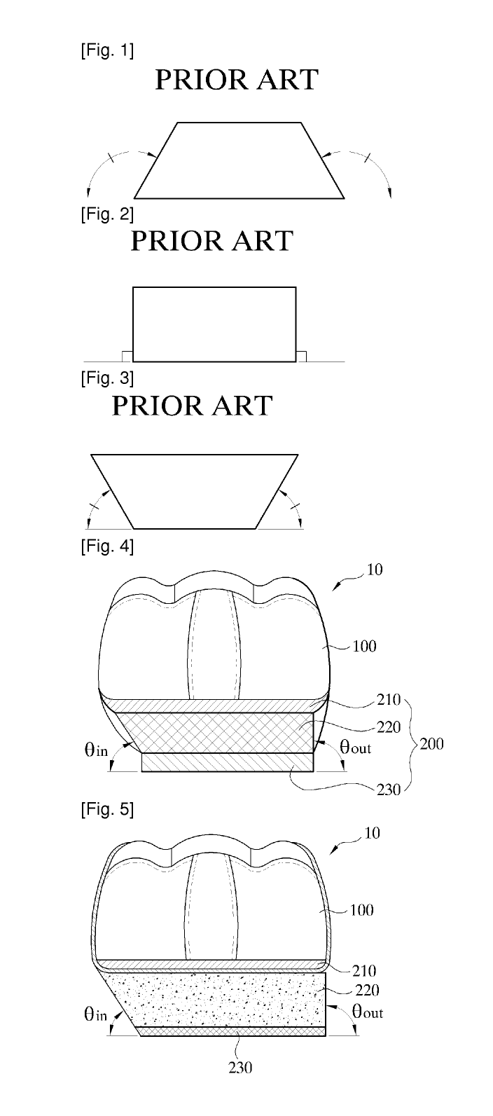

### EXPLANATION OF ESSENTIAL REFERENCE NUMERALS IN DRAWINGS

10: shoe
100: upper
200: sole
210: insole
220: midsole
230: outsole
231: recess
232: projection
a: vector axis of the ground reaction force
a′: moved vector axis of ground reaction force

### BEST MODE FOR CARRYING OUT THE INVENTION

### Embodiment 1

As shown in FIGS. 4 and 5, according to the first preferred embodiment of the present invention, in order to correct bow legs or knock knees, in case of the insole 210, the outsole 230 or the midsole 220 formed integrally with one of the insole 210 and the outsole 230, an angle of one side of an inner face or an outer face to the ground outside the sole 200 is smaller than an angle of the opposed side to the ground outside the sole 200. That is, the two sides are not parallel to each other. The inclination angle is changeable according to a wearer's degree of bending of legs, and preferably, within a range of 45° to 120°.

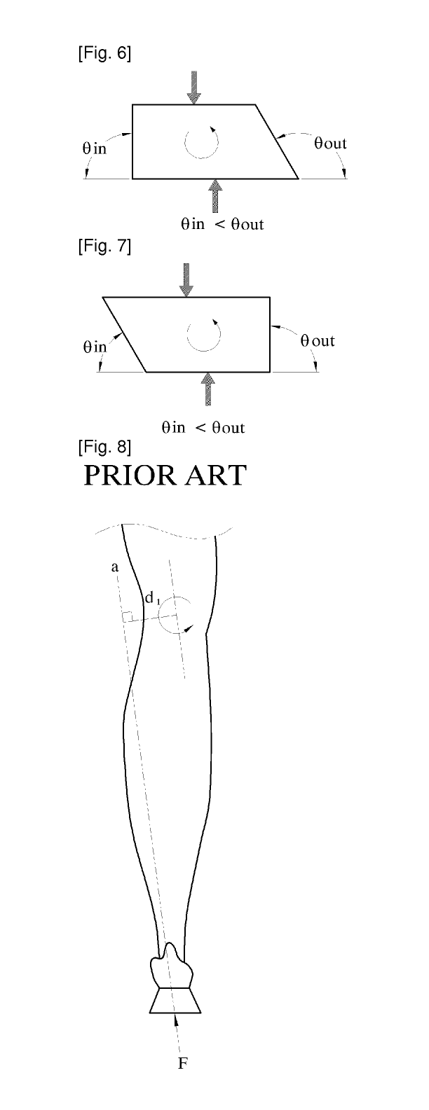

FIG. 6 is a brief longitudinally sectional view showing couple moment occurring by a sole for a wearer, who has bow legs, according to a preferred embodiment of the present invention, and FIG. 7 is a brief longitudinally sectional view showing couple moment occurring by a sole for a wearer, who has bow legs, according to another preferred embodiment of the present invention. As shown in FIGS. 6 and 7, in order to correct bow legs, in case of the insole 210, the outsole 230 or the midsole 220 formed integrally with one of the insole 210 and the outsole 230, an angle (θᵢₙ) of an inner side of the midsole 220 to the ground outside the sole 200 is smaller than an angle (θₒᵤₜ) of an outer side to the ground. In this instance, it is preferable that the angle (θᵢₙ) of the inner side of the sole 200 to the outside ground is an inclination angle within a range of 45° to 120°. Then, couple moment to move the knees toward the center of the body occurs during walking, and thereby, a gap occurs between joints inside the knees. Then, load inside the knees is reduced, and the bow legs are gradually corrected into straight legs.

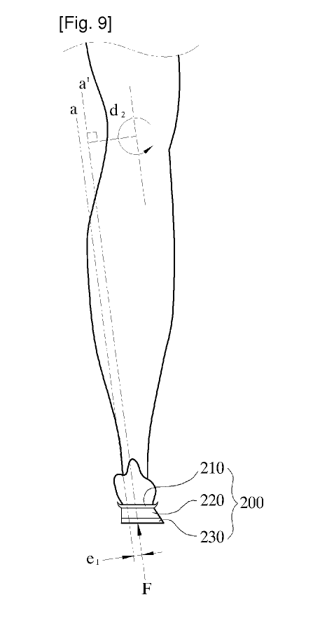

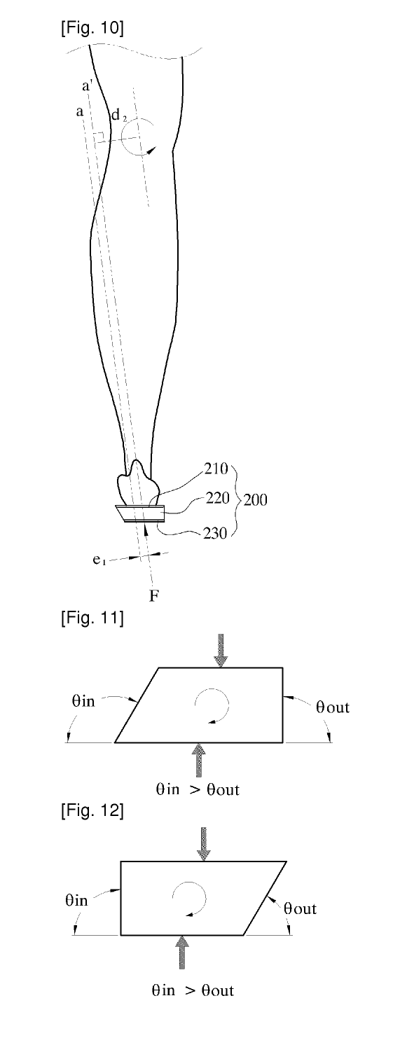

FIG. 8 is a brief view showing abduction moment of the conventional shoe, FIG. 9 is a brief view showing abduction moment of the shoe having the insole and the midsole according to the present invention, and FIG. 10 is a brief view showing abduction moment of the shoe having the insole and the midsole according to the present invention. As shown in FIG. 8, abduction moment occurring by the conventional shoe can be obtained by the following mathematical formula.

M₁=d₁×F, [Formula 1]
wherein M₁ is abduction moment, d₁ is a vertical distance between the center of the knee and a vector axis (a) of the ground reaction force, and F is the ground reaction force occurring from the ground during walking.

As shown in FIGS. 9 and 10, the abduction moment occurring by the shoe 10 according to the present invention can be obtained by the following mathematical formula 2.

M₂=d₂×F=(d₁−e₁)×F, [Formula 2]
wherein M₂ is abduction moment, d₂ is a vertical distance between the center of the knee and a moved vector axis (a′) of the ground reaction force, d₁ is a vertical distance between the center of the knee and a vector axis (a) of the ground reaction force, e1 is a vertical distance between the vector axis (a) of the ground reaction force and the moved vector axis (a′) of the ground reaction force, and F is the ground reaction force occurring from the ground during walking.

As shown in the formula 1 and the formula 2, because M₂ is smaller in abduction moment than M₁, rotational force to move the knee and the lower leg outwardly to thereby relieve the pain inside the anterior knee and correct the bow legs.

FIG. 11 is a brief longitudinally sectional view showing couple moment occurring by a sole for a wearer, who has knock knees, according to a preferred embodiment of the present invention, and FIG. 12 is a brief longitudinally sectional view showing couple moment occurring by a sole for a wearer, who has knock knees, according to another preferred embodiment of the present invention. As shown in FIGS. 11 and 12, in order to correct knock knees, an angle (θₒᵤₜ) of the outer side of the shoe 10 to the ground outside the sole 200 is smaller than an angle (θᵢₙ) of the inner side to the ground outside the sole 200. In this instance, it is preferable that the angle (θₒᵤₜ) of the outer side of the shoe to the outside ground is within a range of 45° to 120°. Then, couple moment, which moves the knees outwardly from the body, occurs during walking, and hence, a gap occurs between the inside joints of the knees. Then, load inside the knees is reduced, and the knock knees are gradually corrected into straight legs.

In this instance, if the angles (θᵢₙ and θₒᵤₜ) of the inner side and the outer side of the shoe 10 to the ground are less than 45° or more than 120°, it is difficult to form normal shoes 10. The inclination angles (θᵢₙ and θₒᵤₜ) are changeable according to the wearer's degree of bending of legs. Moreover, in case that a normal person wears the shoes, the inclination angles (θᵢₙ and θₒᵤₜ) to the ground are about 90 to thereby prevent deformation of the legs. Since one side of the sole 200 is inclined at the predetermined angle (θᵢₙ or θₒᵤₜ), when weight is applied to the sole 200, the traditional treatment method can be naturally realized by a geometric shape of the sole 200.

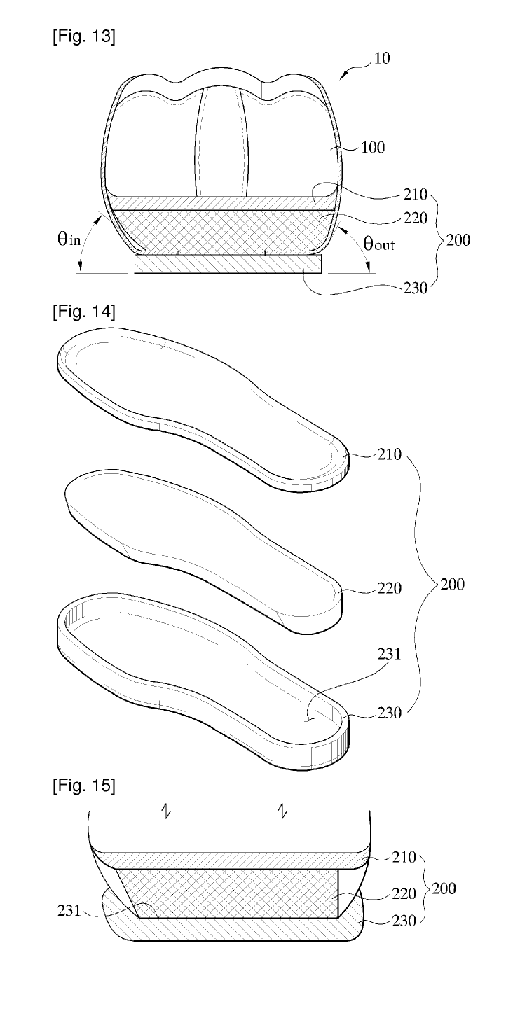

FIG. 13 is a longitudinally sectional view of a shoe including a functional sole having an insole and a midsole for curing a degenerative knee disease according to a modification of the first preferred embodiment of the present invention. As shown in FIG. 13, the insole 210 of the sole 200, or the insole 210 and the midsole 220, which are formed integrally with each other, can be formed round at both sides. Furthermore, the upper 100 can be joined between the outsole 230 and the midsole 220.

### Embodiment 2

FIG. 14 is an exploded perspective view of a sole according to a second preferred embodiment of the present invention, and FIG. 15 is a longitudinally sectional view of the sole according to the second preferred embodiment of the present invention. The insole 210 of the sole 200, or the insole 210 and the midsole 220, which are formed integrally with each other, according to the second preferred embodiment of the present invention generally have structures similar to those of the first preferred embodiment.

However, the outsole 230 includes a recess 231 of a predetermined depth formed on one side of an upper face thereof along the outer circumferential surface of the sole 200. It is preferable that the height of the recess 231 is smaller than the thickness of the midsole 220. Additionally, it is preferable that the recess 231 is as wide as a portion of a lower part of the midsole 220 can be inserted thereto.

### Embodiment 3

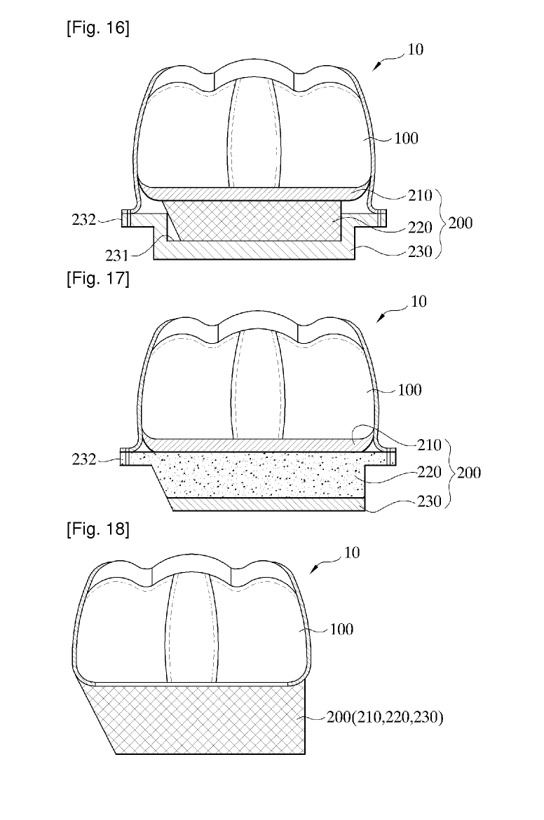

FIG. 16 is a longitudinally sectional view of a shoe according to a third preferred embodiment of the present invention, and FIG. 17 is a longitudinally sectional view of a shoe according to a modification of the third preferred embodiment of the present invention. The shoe 10 according to the third preferred embodiment generally has a structure similar to that of the first preferred embodiment.

However, a projection 232 is formed along the outer circumferential surface of an upper face of the outsole 230 of the sole 200 or the outsole 230 and the midsole 220, which are formed integrally with each other, so that the sole 200 can be joined with the upper 100 smoothly. That is, the projection 232 is formed along the outer circumferential surface of the upper face of the outsole 230 as shown in FIG. 16 or along the outer circumferential surface of the upper face of the midsole 220 as shown in FIG. 17, whereby the sole 200 can be joined with the upper 100 smoothly.

### Embodiment 4

FIG. 18 is a longitudinally sectional view of a shoe including a sole for curing a degenerative knee disease according to a fourth preferred embodiment of the present invention. The upper 100 of the shoe 10 according to the fourth preferred embodiment of the present invention has the same structure as the first preferred embodiment.

As shown in FIG. 18, according to the fourth preferred embodiment of the present invention, the sole 200 includes the insole 210, the midsole 220 and the outsole 230, which are formed integrally with one another, and the whole structure of the sole 200 has the same structure as the insole 210, the outsole 230, or the midsole 220, which is formed integrally with one of the insole 210 and the outsole 230, of the first preferred embodiment.

### MODE FOR THE INVENTION

Reference will be now made in detail to the preferred embodiment of the present invention with reference to the attached drawings.

<Structure of a Shoe Including a Functional Sole to Cure Degenerative Knee Disease>

FIG. 4 is a longitudinally sectional view showing a shoe including a functional sole having an insole and a midsole for curing a degenerative knee disease according to a first preferred embodiment of the present invention, and FIG. 5 is a longitudinally sectional view showing a shoe including a functional sole having an insole and a midsole for curing a degenerative knee disease according to the first preferred embodiment of the present invention. As shown in FIGS. 4 and 5, the shoe 10 according to the present invention includes a sole 200 having an insole 210, a midsole 220 and an outsole 230, and an upper 100. The sole 200 which has distinct features in the structure of the shoe will be described in detail as follows.

### INDUSTRIAL APPLICABILITY

As described above, it is to be appreciated that those skilled in the art can change or modify the embodiments in other various forms without departing from the technical spirit and scope of the present invention. While the present invention has been described with reference to the particular illustrative embodiments, it is not to be restricted by the embodiments but only by the appended claims. It is apparent that all changes or modifications derived from the meaning, scope and equivalent concept of the appended claims are within the scope of the present invention.

What is claimed is:

1. A shoe having a functional sole for degenerative osteoarthritis of a knee joint, which includes a sole having at least one of an insole, a midsole and an outsole, and an upper, characterized in that, in longitudinal section of the insole, the outsole or the midsole formed integrally with one of the insole and the outsole, both sides of an inner face and an outer face thereof are not parallel with each other, and
that an angle of one of both sides of the inner face or the outer face to the ground outside the sole is smaller than an angle of the opposed side to the ground outside the sole.

2. The shoe according to claim 1, characterized in that, in longitudinal section of the insole, the outsole or the midsole formed integrally with one of the insole and the outsole, in order to correct bow legs, an angle of the inner side to the ground outside the sole is smaller than an angle of the outer side to the ground outside the sole.

3. The shoe according to claim 1, characterized in that, in longitudinal section of the insole, the outsole or the midsole formed integrally with one of the insole and the outsole, in order to correct knock knees, an angle of the outer side to the ground outside the sole is smaller than an angle of the inner side to the ground outside the sole.

4. The shoe according to claim 1, characterized in that the insole, the outsole or the midsole formed integrally with one of the insole and the outsole, is changeable in inclination angle and thickness according to a wearer's conditions.

5. The shoe according to claim 1, characterized in that the outsole includes a recess formed on one side of an upper face thereof, and the insole or the insole and the midsole, which are formed integrally with each other, is inserted into the recess of the outsole.

6. The shoe according to claim 1, characterized in that the outsole of the sole or the outsole and the midsole, which are formed integrally with each other, includes a projection formed along the outer circumferential surface of the upper face thereof, so that the sole can be joined with the upper.

7. The shoe according to claim 1, characterized in that, in case of the insole, the outsole or the midsole formed integrally with one of the insole and the outsole, the angle of the inner side to the ground outside the sole for correcting bow legs and the angle of the outer side to the ground outside the sole for correcting the knock knees are within a range of 45° to 120°.

8. The shoe according to claim 1, characterized in that the upper is constructed in such a way as to prevent exposure of the insole and the midsole to the outside.
